(19) United States
(12) Patent Application Publication
(10) Pub. No.: US 2011/0275729 A1
(43) Pub. Date: Nov. 10, 2011

(54) PROCESS FOR CONTROLLED DEGRADATION OF POLYHYDROXYALKANOATES AND PRODUCTS OBTAINABLE THEREFROM

(75) Inventors: Mariastella Scandola, Bologna (IT); Laura Mazzocchetti, Montecopiolo (IT); Maria Letizia Focarete, Bologna (IT); Michal Kawalec, Cueszyn (PL); Piotr Kurcok, Gliwice (PL); Grazyna Adamus, Gliwice (PL); Marek Kowalczuk, Zabrze (PL)

(73) Assignees: INSTITUTE OF POLYMERS AND CARBON MATERIALS, 41-800 Zabrze, Silesia (PL); ALMA MATER STUDIORUM - UNIVERSITA' DI BOLOGNA, 40126 Bologna (IT)

(21) Appl. No.: 13/122,686

(22) PCT Filed: Oct. 15, 2008
(86) PCT No.: PCT/IT2008/000646

§ 371 Date: May 10, 2011

(51) Int. Cl. C08G 63/06; C07D 301/02; C08G 63/91; C07C 69/675; C08J 9/08

(52) U.S. Cl. 521/79; 560/179; 521/92; 549/518

(57) ABSTRACT

The present invention relates to a process for producing polyhydroxyalkanoate oligomers and/or polymers of reduced molecular weight, which comprises reacting at least one polyhydroxyalkanoate (PHA) with at least one carbonate salt at a temperature of from 50° C. to 300° C., preferably from 120° C. to 200° C. The above reaction allows a controlled degradation of the PHA chains which yields oligomers and/or polymers having a controlled molecular weight, which can be modulated in view of the specific application for which the oligomers and/or polymers are intended. Moreover, during the reaction carbon dioxide evolves which produces a foamed material which can be easily processed on an industrial scale. Additionally, the above oligomers and/or polymers, having an end-group bearing a double bond C═C, particularly a crotonate end-group for PHB, can be subjected to subsequent modifications to obtain a wide variety of functional end-groups, for instance carboxyl, carboxylate, hydroxyl, dihydroxyl, oxirane ring, halogen atom. Moreover, the low molecular weight PHAs may be used in the area of controlled delivery systems in agro-chemistry, in the cosmetic industry, in medicine in the form of nano- or microspheres, in household products and in coating systems.

### BACKGROUND OF THE INVENTION

The present invention relates to a process for controlled degradation of polyhydroxyalkanoates and to products obtainable therefrom.

Polyhydroxyalkanoates (PHAs) are polyesters which are produced in nature by bacterial fermentation of sugar or lipids. Synthetic routes are also available. Depending on the molecular weight and, in the case of copolymers, on the specific combination of comonomers, the properties of PHAs may vary from thermoplastic to elastomeric. The mechanical properties and biocompatibility of PHAs can also be changed by blending PHAs with other polymers, enzymes and inorganic materials, to make them suitable for a wide range of applications, particularly in the biomedical field.

One of the most studied PHAs is poly(3-hydroxybutyrate) (PHB), which is widely distributed in nature. For instance, the high molecular weight isotactic PHB is a storage material used by bacteria in nutrition-limited environments as an energy and carbon source. Synthetic analogues of this biodegradable natural polymer of potential industrial importance are obtainable by direct copolymerization of epoxides with carbon monoxide and via ring-opening polymerization of β-butyrolactone (4-methyl-2-oxetanone) to isotactic, atactic (a-PHB) and syndiotactic poly-3-hydroxybutyrate. Short chain PHB, regardless of its tacticity, may be prepared from high molecular weight PHB by high temperature thermal degradation, which proceeds according to a random polymer-chain scission mechanism via intramolecular stereoselective cis-elimination with the formation of oligomers containing trans-crotonate end groups, as the main products. The random chain scission by cis-elimination mechanism has been considered as the general pathway of PHB thermal degradation up to now.

More recently, in International patent application WO 2007/107808, filed in the name of the Applicants, it is described a method for controlling thermal degradation of anionically terminated polymers, particularly of PHAs, by protonating or alkylating said anionic moieties, particularly carboxylate groups. Moreover, it is described the preparation of polymeric blends having controlled thermal stability by mixing PHAs with polymers having an anionically activated moiety. As reported in the above patent application, these inventions are mainly based on the finding that PHAs can undergo chain scission, at moderate temperatures, which results in polymer degradation, via an E1cB elimination mechanism. This elimination mechanism involves the anionic moieties of a polymer which abstracts the acidic proton at C2 position of PHA with the generation of a carbanion, which undergoes the elimination reaction, leading to chain scission.

The Applicants have also tried to achieve a controlled degradation of PHB by reaction with a carboxylate salt, specifically an acetate, at relatively low temperatures (150° C.-170° C.), as described in the article by Kawalec, M. et al, published in. Biomacromolecules, Vol. 8, No. 4, 2007, pages 1053-1058. The results were not satisfactory, since the reaction of PHB with an acetate yields waxy solids or very viscous liquids, which cannot be easily processed, making them unsuitable for applications on a large scale. Moreover, from the reaction mixture the formed acetic acid is evolved which, besides being corrosive, can interfere with the degradation reaction, therefore an effective control of the reaction itself is practically unfeasible.

### SUMMARY OF THE INVENTION

The Applicants have now found that PHAs can be reacted with a carbonate salt so as to achieve a controlled degradation of the PHA chains which yields oligomers and/or polymers having a controlled molecular weight, which is reduced with respect to that of the starting polymers and which can be modulated in view of the specific application for which the products are intended. Moreover, during the reaction carbon dioxide evolves which produces a foamed material which can be easily processed on an industrial scale. Contrary to a compact bulk material which is difficult to handle, the presence of porosity allows, for instance, to obtain a homogeneous powder by grinding or simply crashing the foamed material. In addition, the foamed material can be much more easily solubilized than its bulk analogue. Moreover, the degradation reaction can be carried out as a continuous process, e.g. by means of an extruder, with outstanding advantages for an industrial application.

Additionally, the Applicants have found that the so obtained oligomers or polymers, having an end-group bearing a double bond C═C, e.g. a crotonate end-group for PHB, can be subjected to subsequent modifications to obtain a wide variety of functional end-groups, for instance hydroxyl, carboxyl or oxirane groups by oxidation of the above double bonds. The controlled molecular weight and the presence of double bonds and/or other functional groups as terminal groups make the above oligomers or polymers particularly suitable as macromers (building blocks) for the synthesis and/or modification of polymers, particularly of biodegradable polymers. Moreover, the PHA oligom 1'ers may be used in the area of controlled delivery systems in ago-chemistry, in the cosmetic industry, in medicine in the form of nano- or microspheres, in household products and in coating systems.

Therefore, according to a first aspect, the present invention relates to a process for producing polyhydroxyalkanoate oligomers and/or polymers of reduced molecular weight, which comprises reacting at least one polyhydroxyalkanoate with at least one carbonate salt at a temperature of from 50° C. to 300° C., preferably from 120° C. to 200° C.

According to another aspect the present invention relates to polyhydroxyalkanoate oligomers and/or polymers, said oligomers or polymers being in the form of a foamed material and having a weight average molecular weight (Mw) of from 100 to 100,000, preferably from 1,200 to 25,000. The weight average molecular weight (Mw) can be determined by Size Exclusion Chromatography (SEC) according to known techniques or by mass spectrometry (MALDI TOF and/or ESI MS).

According to another aspect, the present invention relates to polyhydroxyalkanoate oligomers or polymers, said oligomers or polymers having a weight average molecular weight (Mw) of from 100 to 100,000, preferably from 1,200 to 25,000 and having, as first end-group, a carboxyl or carboxylate group and, as second end-group, a functional group selected from: carboxyl, carboxylate, hydroxyl, dihydroxyl, oxirane ring, halogen atom.

### DETAILED DESCRIPTION OF THE INVENTION

As regards the polyhydroxyalkanoates, they are preferably polymers containing repeating units having the following formula:

—O—C(R¹R²)—C(HR³)—C(O)— (I)

wherein:
R¹, R² and R³, equal or different from each other, are selected from: —H, C₁-C₈ alkyl, C₂-C₈ alkenyl, C₆-C₁₀ aryl, said alkyl, alkenyl and aryl groups being optionally substituted by at least one functional group selected from carboxyl, hydroxyl, halogen and alkoxyl groups.

The above polymers may be homopolymers, copolymers or terpolymers. In the case of copolymers or terpolymers, they may be formed by different repeating units corresponding to formula (I), or they may be formed by at least one repeating unit of formula (I) with at least one repeating unit deriving from comonomers able to copolymerize with 3-hydroxyalkanoates, for examples lactones or lactams. In the latter case, the repeating units of formula (I) are preferably present in an amount of at least 5% by mole with respect to the total moles of repeating units.

Particularly preferred repeating units of formula (I) are selected from: 3-hydroxybutyrate, 3-hydroxyvalerate, 3-hydroxyhexanoate, 3-hydroxyoctanoate, 3-hydroxyundec-10-enoate, or combinations thereof.

Particularly preferred PHAs are: poly-3-hydroxybutyrate (P3HB), poly-3-hydroxyvalerate (PHV), poly-3-hydroxyhexanoate (PHH), poly-3-hydroxyoctanoate (PHO), poly(3-hydroxybutyrate-co-3-hydroxyvalerate) (PHBV), poly(3-hydroxybutyrate-co-3-hydroxyhexanoate) (PHBH), poly(3-hydroxybutyrate-co-4-hydroxybutyrate), poly(3-hydroxyoctanoate-co-3-hydroxyundec-10-enoate) (PHOU) copolymers.

As regards the carbonate salt, it can be selected from salts of carbonic acid, particularly carbonates, bicarbonates or mixtures thereof, either anhydrous or in a hydrated form. Counterions may be selected for instance from cations of: alkali metals, e.g. sodium, potassium; alkaline-earth metals, e.g. magnesium, calcium.

The relative amount of the reactants can vary within wide ranges. Generally, the molar ratio between the PHA and the carbonate salt may be within a range from 1000:1 3HA units/salt to 10:1 3HA units/salt, preferably from 500:1 3HA units/salt to 10:1 3HA units/salt.

According to a preferred embodiment, the reaction between the PHAs and the carbonate salt is carried out by thy-mixing the reactants and then by heating the mixture from 50° C. to 300° C., preferably from 120° C. to 200° C. The reaction is preferably carried out at atmospheric pressure, so as to favour the evolution of carbon dioxide and therefore the foaming of the resulting material. The reaction time may be varied within wide limits, mainly depending on the molecular weight that is required for the oligomers or polymers to be produced. Indicatively, the reaction time may vary from 20 sec to 1.5 hours, preferably from 30 sec to 1 hour. Besides the temperature, a parameter that influences the reaction rate is the amount of carbonate and the nature of the counterion in the carbonate salt: bulky cations usually increase the reaction rate. For instance potassium carbonate reacts, in the same reaction conditions, more quickly than sodium carbonate. Moreover, the reaction rate depends also on the effectiveness of the mechanical means used for mixing the reactants. In this respect, the mixing apparatus may be selected from those commonly used for solventless reactions. Particularly preferred are extruders, e.g. single-screw extruders or double-screw extruders, which, besides being very efficient in mixing the reactants (reactive blending), operate continuously and allow an accurate control of the reaction temperature. However, periodic or flow reactors can also be used.

At the end of the reaction, the PHA oligomers or polymers are obtained in the form of a foamed material, which can be easily processed to be used as such or for subsequent reactions. For instance, the foamed material—if brittle—can be grinded according to known techniques, e.g. by foam crashing through compression followed—if required—by ball mill grinding.

The so obtained PHA oligomers or polymers have, as first end-group, a carboxylate end-group, while the second end-group is a functional group containing a double bond C═C, particularly of formula R¹R²C═CR³—COO—, wherein R¹, R² and R³ are defined as above, e.g. for PHB a crotonate group of formula: CH₃—CH═CH—COO—.

The presence of a double bond C═C in the terminal unit allows to use the oligomers or polymers according to the present invention as macromers in radical polymerization processes (free-radical or controlled such as ATRP and/or RAFT), for instance for the synthesis and/or modification of polymers.

Moreover, as indicated above, when required, the terminal double bonds C═C can be subjected to subsequent modifications to obtain a wide variety of functional end-groups, for instance carboxyl, carboxylate, hydroxyl, dihydroxyl, oxirane ring of formula R¹R²C(O)CR³—COO— (wherein R¹, R² and R³ have the same meaning as indicated above), by oxidation of the above double bonds.

The oxidation can be carried out by means of an oxidizing agent, e.g. an inorganic or organic peroxide, peracid or persalt. Suitable oxidizing agents are, for instance: potassium permanganate, chromates, dichromates, Jones reagent and m-chloroperbenzoic acid. The oxidation reaction is preferably carried out using as reaction medium at least one solvent where both the oligomers or polymers and the oxidizing agent are at least partially soluble. Suitable solvents are, for instance: halogenated hydrocarbons, e.g. chloroform, dichloromethane.

Alternatively, the terminal double bonds C═C can be subjected to an addition reaction with a hydrogen halides, particularly HBr or HI, to introduce a halogen atom into the end-group. The resulting modified oligomers or polymers can be useful as macromers in ATRP (Atom Transfer Radical Polymerization) reactions.

The controlled molecular weight and the presence of double bonds and/or other functional groups as terminal groups make the above oligomers or polymers particularly suitable as macromers (building blocks) for the synthesis and/or modification of polymers, particularly of biodegradable polymers. Moreover, the PHA oligomers or polymers may be used in the area of controlled delivery systems in agro-chemistry, in the cosmetic industry, in medicine in the form of nano- or microspheres, in household products and in coating systems.

The following working examples are given to better illustrate the invention, but without limiting its scope.

### Example 1

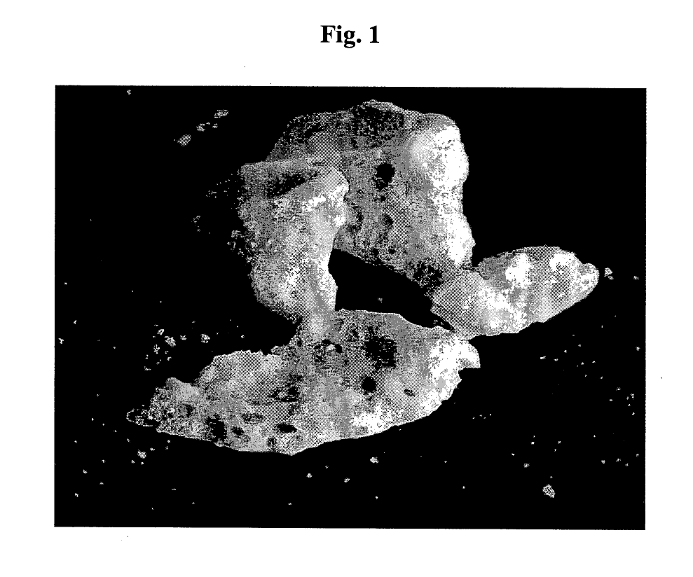

30 g of poly([R]-3-hydroxybutyrate) (PHB) (Mw=431000; Mw/Mn=3) in form of powder dry-mixed with 3 g of anhydrous sodium carbonate (POCh Gliwice) (0.944 mmol of the salt/g of PHB) was thermally treated in a single screw extruder with simultaneous mixing for about 2 minutes with maximum temperature of 170° C. (measured in plasticizing zone). Resulting material was a white brittle foam. SEC analysis, according to conventional polystyrene (PS) calibration, gave Mw=5900 and Mw/Mn=3. A photograph of the resulting foamed material is reported in the enclosed FIG. 1.

For comparison the PHB sample was thermally treated in the same single screw extruder without adding the carbonate: 30 g of the same PHB from Biomer (Mw=431000; Mw/Mn=3) in form of powder was treated in the same extruder with simultaneous mixing for about 2 minutes with maximum temperature of 170° C. (measured in plasticizing zone). The resulting stiff material subjected to SEC analysis according to conventional PS calibration gave Mw=345000 and Mw/Mn=3. In both cases ¹H NMR analysis confirmed the presence of crotonate terminal groups, while no crotonic acid proton signals were observed.

It is to be noted that grinding of the carbonate thermally treated polymer is much easier than grinding the material obtained by mere thermal degradation.

### Example 2

30 g of PHB from Biomer (Mw=431000; Mw/Mn=3) in form of powder was dry-mixed with 6 g of anhydrous sodium carbonate (POCh Gliwice) and thermally treated in a single screw extruder with simultaneous mixing for about 2 minutes with maximum temperature of 150° C. (measured in plasticizing zone). The resulting foamed material was subjected to SEC analysis which gave Mw=39000 and Mw/Mn=4. ¹H NMR analysis confirmed the presence of crotonate terminal groups while no signals of crotonic acid were observed.

### Example 3

20 g of poly([R]-3-hydroxybutyrate) (PHB) from Biomer (Mw=431000, Mw/Mn=3) in form of powder was dry-mixed with 2.608 g of anhydrous potassium carbonate (POCh Gliwice) (0.944 mmol of the salt/g of PHB) and thermally treated in a single screw extruder with simultaneous mixing for about 2 minutes with maximum temperature of 170° C. (measured in plasticizing zone). The resulting material, after cooling, formed a white brittle foam. SEC analysis according to conventional calibration on PS gave Mw=3900 and Mw/Mn=2.

### Example 4 (Comparative)

20 g of poly([R]-3-hydroxybutyrate) (PHB) from Biomer (Mw=431000, Mw/Mn=3.2) in form of powder was dry-mixed with 1.852 g of anhydrous potassium acetate (AcOK) (Fluka) (0.944 mmol of the salt/g of PHB) and thermally treated in a single screw extruder with simultaneous mixing for about 2 minutes with maximum temperature of 170° C. (measured in plasticizing zone). The resulting material was a honey-like dark brown very viscous liquid. SEC analysis according to conventional calibration on PS gave Mw=2900, and Mw/Mn=2. It is to be noted that the resulting oligomer is very difficult to be extracted from the extruder and subsequently processed. Moreover, it is worth noting that the amount cation mols/salt moll in AcOK is only half the amount in the corresponding carbonate, which means that carbonates are degradation agents much milder than acetates, leading to a better control of the degradation reaction.

### Example 5

10 g of PHBV (poly([R]-3-hydroxybutyrate-co-valerate)) from Aldrich (Mw=690000, Mn=280000, Mw/Mn=2.45, 12 mol % of valerate units) in form of powder was dry-mixed with 1 g of anhydrous sodium carbonate (POCh Gliwice) and thermally treated in a single screw extruder with simultaneous mixing for about 2 minutes with maximum temperature of 170° C. (measured in plasticizing zone). The resulting material was a brittle foam. SEC analysis according to conventional calibration on PS gave Mw=10000, Mn=4000 and Mw/Mn=2.5.

For comparison a PHBV sample was thermally treated in the same single screw extruder without adding the carbonate: 20 g of the same PHBV from Aldrich (Mw=690000, Mn=280000, Mw/Mn=2.45, 12 mol % of valerate units) in form of powder was thermally treated in the same extruder with simultaneous mixing for about 2 minutes with maximum temperature of 170° C. (measured in plasticizing zone). The resulting material was in form of a film. SEC analysis according to conventional calibration on PS gave Mw=680000, Mn=275000 and Mw/Mn=2.47. In both cases ¹H NMR analysis confirmed the presence of unsaturated terminal groups.

### Example 6

1 g of PHBV (poly([R]-3-hydroxybutyrate-co-valerate)) from Aldrich (Mw=690000, Mw/Mn=2.45, 12 mol % of valerate units) in form of powder was dry-mixed with 0.10 g of anhydrous sodium carbonate (POCh Gliwice) (0.944 mmol of the salt/g of PHBV; 1.887 meq of Na⁺/g of PHBV). A sample of 100 mg of the mixture was pressed at room temperature in form of pellet. Then the pellet was isothermally treated in an oven at a temperature of 170° C. for 1 hour. The resulting material was in form of light brown foamed wax. SEC analysis revealed Mw=4400, Mw/Mn=2.5.

### Example 7

1 g of PHBV (poly([R]-3-hydroxybutyrate-co-valerate)) from Aldrich (Mw=690000, Mw/Mn=2.45, 12 mol % of valerate units) in form of powder was dry-mixed with 0.1304 g of anhydrous potassium carbonate (POCh Gliwice) (0.944 mmol of the salt/g of PHBV; 1.887 meq of K⁺/g of PHBV). A sample of 100 mg of the mixture was pressed in form of pellet. Then the pellet was isothermally treated in an oven at a temperature of 170° C. for 1 hour. The resulting material was in form of brownish slightly foamed wax. SEC analysis revealed Mw=550, Mw/Mn=1.3.

### Example 8 (Comparative)

1 g of PHBV (poly([R]-3-hydroxybutyrate-co-valerate)) from Aldrich (Mw=690000, Mw/Mn=2.45, 12 mol % of valerate units) in form of powder was dry-mixed with 0.0926 g of potassium acetate (Aldrich) (0.944 mmol of the salt/g of PHBV; 0.944 meq of K⁺/g of PHBV). A sample of 100 mg of the mixture was pressed in form of pellet. Then the pellet was isothermally treated in an oven at a temperature of 170° C. for 1 hour. The resulting material was in form of dark brown wax. SEC analysis revealed Mw=1500, Mw/Mn=2.1.

### Example 9 (Comparative)

1 g of PHBV (poly([R]-3-hydroxybutyrate-co-valerate)) from Aldrich (Mw=690000, Mw/Mn=2.45, 12 mol % of valerate units) in form of powder was dry-mixed with 0.1852 g of potassium acetate (Aldrich) (1.887 mmol of the salt/g of PHBV; 1.887 meq of K⁺/g of PHBV). A sample of 100 mg of the mixture was pressed in form of pellet. Then the pellet was isothermally treated in an oven at a temperature of 170° C. for 1 hour. The resulting material was in form of dark brown very viscous liquid. SEC analysis revealed Mw=700, Mw/Mn=1.8.

The above Examples 6-7 illustrate the influence of the salt type and cation size on the kinetics of the degradation. Comparing Example 6 with Example 7 it is apparent that a larger cation (K⁺ versus Na⁺) yields a more active salt. Comparing Examples 7 and 9, where counterion and concentration were the same, but the salts were different (carbonate versus acetate), it can be noted that the use of carbonates yields oligomers in form of foam that can be easily processed, and moreover corrosive vapors of acetic acid are avoided.

### Example 10

1 g of PHBV (poly([R]-3-hydroxybutyrate-co-valerate)) from Aldrich (Mw=690000, Mw/Mn=2.45, 12 mol % of valerate units) in form of powder was dry-mixed with 0.10 g of anhydrous sodium carbonate (POCh Gliwice) (0.944 mmol of the salt/g of PHB; 1.887 meq of K⁺/g of PHBV). A sample of 100 mg of the mixture was pressed in form of pellet. Then the pellet was isothermally treated in an oven at a temperature of 170° C. for 2 hours. The resulting material was in form of brownish slightly foamed wax. SEC analysis revealed Mw=550, Mw/Mn=1.6.

By comparing Example 10 with Example 6, it is apparent the influence of the reaction time on the molar mass of the resulting product: the longer the degradation time the lower the molar mass of the resulting oligomer.

### Example 11

1 g of PHBV (poly([R]-3-hydroxybutyrate-co-valerate)) from Aldrich (Mw=690000, Mw/Mn=2.45, 12 mol % of valerate units) in form of powder was dry-mixed with 0.05 g of anhydrous sodium carbonate (POCh Gliwice) (0.472 mmol of the salt/g of PHB; 0.944 meq of Na⁺/g of PHBV). A sample of 100 mg of the mixture was pressed in form of pellet. Then the pellet was isothermally treated in an oven at a temperature of 170° C. for 1 hour. The resulting material was in form of pale yellow solid foam. SEC analysis revealed Mw=6100, Mw/Mn=2.8.

By comparing Example 11 with Example 6, it is apparent the influence of the salt amount on the molar mass of the resulting product: the higher the salt amount the lower the molar mass of the resulting oligomer.

### Example 12

2 g of PHB (poly([R]-3-hydroxybutyrate)) from Biomer (Mw=431000, Mw/Mn=3) in form of powder were mixed with 0.1585 g of anhydrous sodium bicarbonate (POCh Gliwice) (0.9435 mmol of the salt/g of PHB; 0.9435 meq of Na⁺/g of PHB). A sample of 100 mg of the mixture was pressed in form of pellet. Next the pellets have been isothermally treated at temperature of 200° C. for 5 min. The resulting material was a white brittle foam. SEC analysis revealed Mw=4150, Mw/Mn=2.6, mass at peak maximum Mp=4900.

### Example 13

2 g of PHB (poly([R]-3-hydroxybutyrate)) from Biomer (Mw=431000, Mw/Mn=3) in form of powder were mixed with 0.317 g of anhydrous sodium bicarbonate (POCh Gliwice) (1.887 mmol of the salt/g of PHB; 1.887 meq of Na⁺/g of PHB). A sample of 100 mg of the mixture was pressed in form of pellet. Next the pellets have been isothermally treated at temperature of 200° C. for 5 min. The resulting material was a white brittle foam. SEC, analysis revealed Mw=4200, Mw/Mn=2.35, mass at peak maximum Mp=3700.

### Example 14

2 g of PHB (poly([R]-3-hydroxybutyrate)) from Biomer (Mw=431000, Mw/Mn=3) in form of powder were mixed with 0.2 g of anhydrous sodium carbonate (POCh Gliwice) (0.9435 mmol of the salt/g of PHB; 1.887 meq of Na⁺/g of PHB). A sample of 100 mg of the mixture was pressed in form of pellet. Next the pellets have been isothermally treated at temperature of 200° C. for 5 min. The resulting material was a white brittle foam. SEC analysis revealed Mw=4300, Mw/Mn=3.88, mass at peak maximum Mp=3450.

### Example 15 (Comparative)

A sample of 100 mg of PHB (poly([R]-3-hydroxybutyrate)) from Biomer (Mw=431000, Mw/Mn=3) in form of powder was taken and pressed in form of pellet. Next the pellets were isothermally treated at temperature of 200° C. for 5 min. The resulting material was a white brittle pellet. SEC analysis revealed Mw=398000, Mw/Mn=2.79, mass at peak maximum Mp=419000.

### Example 16

3.319 g (12.25 mmol) of m-chloroperoxybenzoic acid was added into a reactor containing a solution of 0.5 g of aPHB (atactic poly([R, S]-3-hydroxybutyrate) (Mw=1500) having terminal crotonate groups in 10 ml of methylene chloride. The oxidation reaction was carried out at 30° C. for 48 hours by vigorously stirring the reaction mixture. Then the mixture was washed with 20 ml 5% aqueous solution of sodium carbonate and washed five times with water, acidified with 20 ml of diluted HCl_{(aq)} subsequently. Next it had been washed five times with 20 ml of distilled water until neutral pH-value was reached. The product was isolated by precipitation in cold hexane. The obtained product had terminal 2,3-epoxybutyrate group, Mw=1350.

1. A process for producing polyhydroxyalkanoate (PHA) oligomers and/or polymers of reduced molecular weight, which comprises reacting at least one PHA with at least one carbonate salt at a temperature of from 50° C. to 300° C., preferably from 120° C. to 200° C.

2. The process according to claim 1, wherein the at least one PHA contains at least one repeating unit having the following formula:
—O—C(R¹R²)—C(HR³)—C(O)— (I)
wherein:
R¹, R² and R³, equal or different from each other, are selected from: —H, C₁-C₈ alkyl, C₂-C₈ alkenyl, C₆-C₁₀ aryl, said alkyl, alkenyl and aryl groups being optionally substituted by at least one functional group selected from carboxyl, hydroxyl, halogen and alkoxyl groups.

3. The process according to claim 2, wherein the at least one PHA is a homopolymer, copolymer or terpolymer formed: (i) by different repeating units corresponding to formula (I), or (ii) by at least one repeating unit of formula (I) and by at least one repeating unit deriving from comonomers able to copolymerize with 3-hydroxyalkanoates, the repeating units of formula (I) being present in an amount of at least 5% by mole with respect to the total moles of repeating units.

4. The process according to claim 2, wherein the at least one repeating unit of formula (I) is selected from: 3-hydroxybutyrate, 3-hydroxyvalerate, 3-hydroxyhexanoate, 3-hydroxyoctanoate, 3-hydroxyundec-10-enoate, or combinations thereof.

5. The process according to claim 1, wherein the at least one carbonate salt is selected from carbonates, bicarbonates or mixtures thereof, either anhydrous or in a hydrated form.

6. The process according to claim 1, wherein the at least one carbonate salt has a counterion selected from cations of: alkali metals, e.g. sodium, potassium; alkaline-earth metals, e.g. magnesium, calcium.

7. The process according to claim 1 wherein the molar ratio between the PHA and the carbonate salt is within a range from 1000:1 3HA units/salt to 10:1 3HA units/salt, preferably from 500:1 3HA units/salt to 10:1 3HA units/salt.

8. The process according to claim 1, wherein the reaction is carried out at atmospheric pressure.

9. The process according to claim 1, wherein the reaction time ranges from 20 sec to 1.5 hours, preferably from 30 sec to 1 hour.

10. The process according to claim 1, wherein the reaction is carried out continuously, preferably in an extruder.

11. The process according to claim 1, wherein the reaction is carried out in a periodic or flow reactor.

12. The process according to claim 1, wherein the so obtained oligomers and/or polymers are subsequently subjected to an oxidation reaction.

13. The process according to claim 12, wherein the oxidation reaction is carried out by means of an inorganic or organic peroxide, peracid or persalt.

14. The process according to claim 1, wherein the so obtained oligomers and/or polymers are subsequently subjected to an addition reaction with a hydrogen halide, particularly HBr or HI.

15. Polyhydroxyalkanoate oligomers and/or polymers of reduced molecular weight, said oligomers and/or polymers being in the form of a foamed material and having a weight average molecular weight (Mw) of from 100 to 100,000, preferably from 1,200 to 25,000.

16. Polyhydroxyalkanoate oligomers and/or polymers of reduced molecular weight, said oligomers and/or polymers having a weight average molecular weight (Mw) of from 100 to 100,000, preferably from 1,200 to 25,000 and having, as first end-group, a carboxyl or carboxylate group and, as second end-group, a functional group selected from: carboxyl, carboxylate, hydroxyl, dihydroxyl, oxirane ring, halogen atom.

17. Polyhydroxyalkanoate oligomers and/or polymers according to claim 15, containing at least one repeating unit having the following formula:
—O—C(R¹R²)—C(HR³)—C(O)— (I)
wherein:
R¹, R² and R³, equal or different from each other, are selected from: —H, C₁-C₈ alkyl, C₂-C₈ alkenyl, C₆-C₁₀ aryl, said alkyl, alkenyl and aryl groups being optionally substituted by at least one functional group selected from carboxyl, hydroxyl, halogen and alkoxyl groups.

18. Polyhydroxyalkanoate oligomers and/or polymers according to claim 17, containing: (i) different repeating units corresponding to formula (I), or (ii) at least one repeating unit of formula (I) with at least one repeating unit deriving from comonomers able to copolymerize with 3-hydroxyalkanoates, the repeating units of formula (I) being present in an amount of at least 5% by mole with respect to the total moles of repeating units.

19. Polyhydroxyalkanoate oligomers and/or polymers according to claim 18, wherein the at least one repeating unit of formula (I) is selected from: 3-hydroxybutyrate, 3-hydroxyvalerate, 3-hydroxyhexanoate, 3-hydroxyoctanoate, 3-hydroxyundec-10-enoate, or combinations thereof.

20. A method of using polyhydroxyalkanoate oligomers and/or polymers according to claim 15, comprising a step of using said polyhydroxyalkanoate oligomers and/or polymers as macromers (building blocks) for the synthesis and/or modification of polymers, particularly of biodegradable polymers.
